(19) United States
(12) Patent Application Publication
(10) Pub. No.: US 2008/0093586 A1
(43) Pub. Date: Apr. 24, 2008

(54) Use Of Statistical Copolymers

(76) Inventors: Matthias Koch, Wiebaden (DE); Ralf Anselmann, Luedinghausen-Seppenrade (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/661,035

(22) PCT Filed: Aug. 3, 2005
(86) PCT No.: PCT/EP05/08400

§ 371 Date: Feb. 26, 2007

(30) Foreign Application Priority Data
Aug. 27, 2004 (DE) 10 2004 041 536.6

(51) Int. Cl. B01F 3/12; A61K 8/04; A61Q 19/00

(52) U.S. Cl. 252/589; 424/401; 516/34; 516/87; 524/409

(57) ABSTRACT

The invention relates to the use of statistical copolymers containing at least one structural unit with hydrophobic radicals and at least one structural unit with hydrophilic radicals as dispersants for producing dispersions with an incompatible disperse and continuous phase, in particular, for dispersing particles with a hydrophilic surface in oils, dispersions or powder compositions, containing statistical copolymers and particles with a hydrophilic surface. The invention also relates to methods for producing these statistical copolymers.

The invention relates to the use of random copolymers as dispersants for the preparation of dispersions having incompatible disperse and continuous phases, in particular for the dispersal of particles having a hydrophilic surface in oils, to dispersions or powder compositions comprising random copolymers and particles having a hydrophilic surface, and to processes for the preparation thereof.

Dispersions are used in numerous areas. Thus, paints and coatings, cosmetic and pharmaceutical compositions and cleaning and coating compositions are frequently dispersions of particles in liquids. A significant problem area in the use of dispersions is the tendency of particles to form agglomerates, which can impair the shelf life of the dispersions. There is therefore a constant demand for improved dispersants which ensure stable dispersions.

It is furthermore important for the stability of dispersions that the disperse and continuous phases are compatible. Hydrophilic particles can be dispersed comparatively simply in water using dispersants, but only form dispersions with difficulty with oils. Dispersion assistants which mediate between the phases are usual for compatibilisation of incompatible phases of this type.

However, very high concentrations of these dispersion assistants are in some cases necessary in order to produce stable dispersions. There are hitherto no truly satisfactory dispersants available for the preparation of stable dispersions of highly hydrophilic particles in oils.

There therefore continues to be a demand for dispersants which allow the preparation of stable dispersions having incompatible disperse and continuous phases, in particular dispersions of hydrophilic particles in oils.

Surprisingly, it has now been found that certain copolymers are eminently suitable as dispersants for the preparation of dispersions having incompatible disperse and continuous phases.

The present invention therefore relates firstly to the use of random copolymers containing at least one structural unit having hydrophobic radicals and at least one structural unit having hydrophilic radicals as dispersants for the preparation of dispersions having incompatible disperse and continuous phases.

Particular preference is given here to the use of random copolymers containing at least one structural unit having hydrophobic radicals and at least one structural unit having hydrophilic radicals as dispersants for the dispersal of particles having a hydrophilic surface in oils.

The present invention accordingly furthermore relates to an oily dispersion comprising hydrophilic particles, characterised in that the dispersant present is at least one random copolymer containing at least one structural unit having hydrophobic radicals and at least one structural unit having hydrophilic radicals.

A process according to the invention for the preparation of an oily dispersion of hydrophilic particles is characterised in that random copolymers containing at least one structural unit having hydrophobic radicals and at least one structural unit having hydrophilic radicals are mixed with an oil and hydrophilic particles. In a preferred variant, the random copolymers are initially introduced in an oil, and the hydrophilic particles are subsequently added. In another preferred variant, an aqueous dispersion of hydrophilic particles is mixed (emulsified) with a solution of a random copolymer in a hydrophobic solvent, and the water or both solvents is (are) subsequently removed.

A process according to the invention for the preparation of an aqueous dispersion of hydrophobic particles is characterised in that random copolymers containing at least one structural unit having hydrophobic radicals and at least one structural unit having hydrophilic radicals are mixed with water and hydrophobic particles. In this case, the random copolymers are preferably initially introduced in water, and the hydrophobic particles are subsequently added.

In a preferred embodiment of the present invention, the dispersions, in particular oily dispersions, can be prepared starting from redispersible particles. The present invention therefore likewise relates to corresponding powder compositions comprising hydrophilic particles, characterised in that the hydrophilic particles are coated with at least one random copolymer containing at least one structural unit having hydrophobic radicals and at least one structural unit having hydrophilic radicals.

The powder compositions can be obtained by preparing a dispersion by the above-mentioned processes and subsequently removing the solvent.

The powder compositions according to the invention usually comprise particles having a hydrophilic surface in a proportion by weight of from 20 to 95% by weight, preferably from 30 to 80% by weight, based on the powder composition.

Through the choice of random copolymers comprising at least one monomer having hydrophobic radicals and at least one monomer having hydrophilic radicals, dispersants which facilitate the dispersal of particles having a hydrophilic surface in hydrophobic media and vice versa have now successfully been provided. At the same time, the use of these novel dispersants enables the particles to be isolated from the dispersions as redispersible powder composition in a virtually agglomerate-free manner since the individual particles can be separated off directly with a polymer coating.

The dispersions which can be prepared with the aid of the random copolymers are distinguished by excellent stability. In addition, comparatively small amounts of the copolymers are often sufficient for the preparation of stable dispersions.

In addition, the powder compositions obtainable by this method can be redispersed particularly simply and uniformly.

It is furthermore advantageous that few or no agglomerates of the dispersed particles form in the dispersions according to the invention. In particular, undesired impairment of the transparency of such dispersions in visible light can be substantially avoided if correspondingly small particles are dispersed.

The dispersed particles preferably have an average particle size, determined by dynamic light scattering or transmission electron microscope, of from 3 to 200 nm, in particular from 20 to 80 nm and very particularly preferably from 30 to 50 nm. In specific, likewise preferred embodiments of the present invention, the particle size distribution is narrow, i.e. the variation latitude is less than 100% of the average, particularly preferably at most 50% of the average.

In a further variant of the present invention, the dispersed particles have an average particle size in the range from 500 to 5000 nm. It may likewise be preferred in accordance with the invention to disperse anisotropic particles, particularly preferably platelets having a thickness in the range from 500 to 5000 nm and a diameter in the range from 5 to 100 μm.

The use of the dispersants according to the invention for the dispersal of hydrophilic particles which have a metal (hydr)oxide surface is particularly advantageous, where the metal (hydr)oxide is preferably selected from oxides and hydroxides of silicon, aluminium, magnesium, antimony, cerium, cobalt, chromium, indium, nickel, zinc, titanium, iron, yttrium, tin, zirconium and mixtures thereof. Particles of this type can only be wetted by oils with great difficulty and are therefore regarded as only being dispersible in oils with difficulty using conventional dispersants. It is particularly preferred in accordance with the invention if silica particles or silica-coated particles are dispersed.

Thus, silicon dioxide particles, which can be obtained, for example, by the process described in U.S. Pat. No. 4,911,903, can preferably be employed. The cores here are produced by hydrolytic polycondensation of tetra-alkoxysilanes in an aqueous-ammoniacal medium, where firstly a sol of primary particles is formed, and the resultant SiO₂ particles are subsequently brought to the desired particle size by continuous, controlled metered addition of tetraalkoxysilane. This process enables the production of monodisperse SiO₂ cores having average particle diameters of between 0.05 and 10 μm with a standard deviation of 5%. Corresponding products are commercially available under the trade name Monospher® (Merck).

It is furthermore possible to employ SiO₂ particles which are coated with (semi)metals or with metal oxides which do not absorb in the visible region, such as, for example, TiO₂, ZrO₂, ZnO₂, SnO₂ or Al₂O₃. The production of metal oxide-coated SiO₂ cores is described in greater detail, for example, in U.S. Pat. No. 5,846,310, DE 198 42 134 and DE 199 29 109.

Thus, it is also possible to employ monodisperse particles comprising non-absorbent metal oxides, such as TiO₂, ZrO₂, ZnO₂, SnO₂ or Al₂O₃, or metal-oxide mixtures. Their production is described, for example, in EP 0 644 914. Furthermore, the process according to EP 0 216 278 for the production of monodisperse SiO₂ particles can readily be applied to other oxides with the same result. Tetraethoxysilane, tetrabutoxytitanium, tetrapropoxyzirconium or mixtures thereof are added in one portion to a mixture of alcohol, water and ammonia whose temperature is set precisely to from 30 to 40° C. using a thermostat, and the resultant mixture is stirred vigorously for a further 20 seconds, during which a suspension of monodisperse particles in the nanometer region forms. After a post-reaction time of from 1 to 2 hours, the cores are separated off in a conventional manner, for example by centrifugation, washed and dried.

Other silica particles which can likewise advantageously be dispersed by the method described in the present invention are commercially available, for example, under the trade names Ronaspher® (Merck) or Aerosil® (Degussa). In general, silica particles of virtually any shape can be dispersed by means of the polymers to be employed in accordance with the invention. Thus, the particles can, for example, be spherical, hollow, porous or nonporous platelet-shaped, rod-shaped, platelet-shaped or amorphous and thus without a specific geometric spatial shape.

Corresponding particles can be employed, for example, as filling materials or for coating.

In a preferred embodiment of the present invention, the particles to be dispersed can furthermore be capsules. Capsules particularly preferably to be employed in accordance with the invention have walls which can be obtained by a sol-gel process, as described in the applications WO 00/09652, WO 00/72806 and WO 00/71084. Preference is in turn given here to capsules whose walls are built up from silica gel (silica; undefined silicon oxide hydroxide). The production of corresponding capsules is known to the person skilled in the art, for example from the cited patent applications, the disclosure content of which is expressly also part of the subject-matter of the present application. Particular preference is given here to capsules which contain UV filters. Both for UVA and UVB filters, there are many proven substances which are known from the specialist literature, for example benzylidenecamphor derivatives, such as 3-(4′-methylbenzylidene)-dl-camphor (for example Eusolex® 6300), 3-benzylidenecamphor (for example Mexoryl® SD), polymers of N-{(2 and 4)-[(2-oxoborn-3-ylidene)methyl]benzyl}acrylamide (for example Mexoryl® SW), N,N,N-trimethyl-4-(2-oxoborn-3-yl-idenemethyl)anilinium methylsulfate (for example Mexoryl® SK) or (2-oxoborn-3-ylidene)toluene-4-sulfonic acid (for example Mexoryl® SL), benzoyl- or dibenzoylmethanes, such as 1-(4-tert-butylphenyl)-3-(4-methoxyphenyl)propane-1,3-dione (for example Eusolex® 9020) or 4-isopropyldibenzoylmethane (for example Eusolex® 8020), benzophenones, such as 2-hydroxy-4-methoxybenzophenone (for example Eusolex® 4360) or 2-hydroxy-4-methoxybenzophenone-5-sulfonic acid and its sodium salt (for example Uvinul® MS-40), methoxycinnamic acid esters, such as octyl methoxycinnamate (for example Eusolex® 2292), isopentyl 4-methoxycinnamate, for example as a mixture of the isomers (for example Neo Heliopan® E 1000), salicylate derivatives, such as 2-ethylhexyl salicylate (for example Eusolex® OS), 4-isopropylbenzyl salicylate (for example Megasol®) or 3,3,5-trimethylcyclohexyl salicylate (for example Eusolex® HMS), 4-aminobenzoic acid and derivatives, such as 4-aminobenzoic acid, 2-ethylhexyl 4-(dimethylamino)benzoate (for example Eusolex® 6007), ethoxylated ethyl 4-aminobenzoate (for example Uvinul® P25), phenylbenzimidazolesulfonic acids, such as 2-phenylbenzimidazole-5-sulfonic acid and potassium, sodium and triethanolamine salts thereof (for example Eusolex® 232), 2,2-(1,4-phenylene)bisbenzimidazole-4,6-disulfonic acid and salts thereof (for example Neoheliopan® AP) or 2,2-(1,4-phenylene)bisbenzimidazole-6-sulfonic acid;

and further substances, such as

2-ethylhexyl 2-cyano-3,3-diphenylacrylate (for example Eusolex® OCR),
3,3′-(1,4-phenylenedimethylene)bis(7,7-dimethyl-2-oxobicyclo[2.2.1]-hept-1-ylmethanesulfonic acid and salts thereof (for example Mexoryl® SX),
2,4,6-trianilino-(p-carbo-2′-ethylhexyl-1′-oxy)-1,3,5-triazine (for example Uvinul® T 150) and
hexyl 2-(4-diethylamino-2-hydroxybenzoyl)benzoate (for example Uvinul® UVA Plus, BASF).

The compounds mentioned in the list should only be regarded as examples. It is of course also possible to use other UV filters in the capsules. Preferred compounds having UV-filtering properties are 3-(4′-methylbenzylidene)-dl-camphor, 1-(4-tert-butylphenyl)-3-(4-methoxyphenyl)propane-1,3-dione, 4-isopropyldibenzoylmethane, 2-hydroxy-4-methoxybenzophenone, octyl methoxycinnamate, 3,3,5-trimethylcyclohexyl salicylate, 2-ethylhexyl 4-(dimethylamino)benzoate, 2-ethylhexyl 2-cyano-3,3-diphenylacrylate, 2-phenylbenzimidazole-5-sulfonic acid and the potassium, sodium and triethanolamine salts thereof.

An SiO₂ capsule which contains octyl methoxycinnamate as UV filter is commercially available, for example, under the name Eusolex® UV Pearl™ OMC from Merck KGaA.

It may furthermore be preferred in accordance with the invention to disperse inorganic UV filters. The inorganic UV filters employed for the use according to the invention are preferably nanoparticulate metal oxides. Thus, in particular, titanium dioxide, iron oxides, zinc oxide or also cerium oxides are suitable for use as UV filters, with titanium dioxide being particularly preferred in accordance with the invention as metal oxide since it performs the tasks according to the invention in a particular manner. Titanium dioxide here can be in the form of rutile or anatase or in amorphous form, but preferably in the form of rutile and/or anatase. The preferred primary particle size is in the range from 5 to 50 nm. The primary particles here are preferably round, in particular in the case of anatase, while rutile primary particles frequently occur in needle or spindle shape as far as ovals (“egg-shaped”). However, round rutile primary particles can also be employed in accordance with the invention. Preferred inorganic UV filters here have a silicon dioxide coating, which covers the nanoparticulate metal oxide as completely as possible. It has been found that it is advantageous for the silicon dioxide content, based on the entire nanoparticulate UV protection composition, to be from 5 to 50% by weight, preferably from 8 to 30% by weight and particularly preferably from 12 to 20% by weight. The resultant nanoparticulate UV protection composition usually exhibits a particle size by the Scherrer method in the range from 5 nm to 100 nm, preferably in the range from 8 to 50 nm and particularly preferably below 25 nm. The dimensions of the nanoparticulate UV protection composition, which can be determined under the transmission electron microscope, are usually from 5 to 160 nm in length and from 10 to 70 nm in width. The length is preferably in the range from 30 to 70 nm and the width in the range from 18 to 40 nm. These inorganic UV filters are generally incorporated into cosmetic compositions in an amount of from 0.5 to 20 percent by weight, preferably 2-10%. The use of the copolymers for the dispersal of silica-coated titanium dioxide, which is commercially available, for example, under the name Eusolex® T-AVO (Merck KGaA), is particularly preferred in accordance with the invention.

The particles having a hydrophilic surface are usually dispersed here in a proportion by weight of from 1 to 90% by weight, preferably from 10 to 60% by weight, based on the dispersion.

The dispersant is usually employed in a concentration of from 0.5 to 80% by weight, preferably in a concentration of from 1 to 50% by weight and particularly preferably in a concentration of from 2 to 8% by weight, based on the dispersion as a whole.

The random copolymers preferably to be employed in accordance with the invention exhibit a weight ratio of structural units having hydrophobic radicals to structural units having hydrophilic radicals in the random copolymers in the range from 1:10 to 500:1, preferably in the range from 1:2 to 100:1 and particularly preferably in the range from 1:1 to 10:1. The weight average molecular weight of the preferred random copolymers is in the range from M_{w}=1000 to 1,000,000 g/mol, preferably in the range from 2000 to 50,000 g/mol.

It has been found here that the requirements according to the invention are satisfied in a particular manner by, in particular, copolymers which conform to the formula I 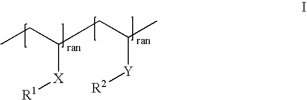
where
X and Y correspond to the radicals of conventional nonionic or ionic monomers, and
R¹ stands for hydrogen or a hydrophobic side group, preferably selected from branched or unbranched alkyl radicals having at least 4 carbon atoms, in which one or more, preferably all, H atoms may have been replaced by fluorine atoms, and
R² stands for a hydrophilic side group, which preferably has one or more phosphonate, phosphate, phosphonium, sulfonate, sulfonium, (quaternary) amine, polyol or polyether radicals, particularly preferably one or more hydroxyl radicals,
ran means that the respective groups in the polymer are arranged in a random distribution,
and where —X—R¹ and —Y—R² may each have a plurality of different meanings within a molecule, and, besides the structural units shown in the formula I, the copolymers may contain further structural units, preferably those with no or with short side chains, such as, for example, C₁₋₄-alkyl.

Particular preference is in turn given here to polymers of the formula I in which X and Y, independently of one another, stand for —O—, —C(═O)—O—, —C(═O)—NH—, —(CH₂)ₙ—, phenyl, naphthyl or pyridyl. Furthermore, polymers in which at least one structural unit contains at least one quaternary nitrogen or phosphorus atom, where R² preferably stands for a —(CH₂)ₘ—(N⁺(CH₃)₂)—(CH₂)ₙ—SO₃⁻ side group or a —(CH₂)ₘ—(N⁺(CH₃)₂)—(CH₂)ₙ—PO₃²⁻, —(CH₂)ₘ—(N⁺(CH₃)₂)—(CH₂)ₙ—O—PO₃²⁻ side group or a —(CH₂)ₘ—(P⁺(CH₃)₂)—(CH₂)ₙ—SO₃⁻ side group, where m stands for an integer from the range from 1 to 30, preferably from the range from 1 to 6, particularly preferably 2, and n stands for an integer from the range from 1 to 30, preferably from the range from 1 to 8, particularly preferably 3, can advantageously be employed.

Random copolymers particularly preferably to be employed can be pre-pared corresponding to the method described in DE 10 2004 004 210.1, in accordance with the following scheme:

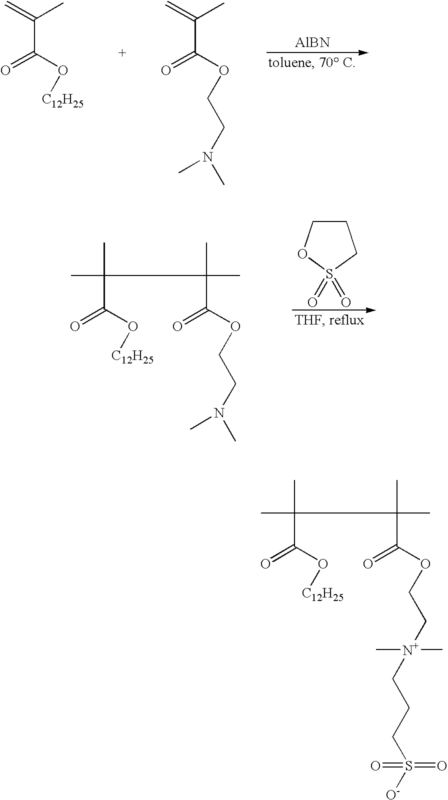

The desired amounts of lauryl methacrylate (LMA) and dimethylaminoethyl methacrylate (DMAEMA) are copolymerised here by known processes, preferably by means of free radicals in toluene by addition of AIBN. A betaine structure is subsequently obtained by known methods by reaction of the amine with 1,3-propane sultone.

In a particularly preferred variant, a copolymer of lauryl methacrylate (LMA) and hydroxyethyl methacrylate (HEMA) is employed. This polymer is likewise preferably copolymerised by free-radical polymerisation of the monomers in toluene by addition of AIBN.

Alternative copolymers preferably to be employed can contain styrene, vinylpyrrolidone, vinylpyridine, halogenated styrene or methoxystyrene, where these examples do not represent a limitation. In another, likewise preferred embodiment of the present invention, use is made of polymers which are characterised in that at least one structural unit is an oligomer or polymer, preferably a macromonomer, where polyethers, polyolefins and polyacrylates are particularly preferred as macromonomers.

It may be further preferred in accordance with the invention for the random copolymers to contain at least one structural unit which has a phosphonium or sulfonium radical.

Furthermore, it may be preferred in accordance with the invention if, besides the at least one structural unit having hydrophobic radicals and the at least one structural unit having hydrophilic radicals, the random copolymers contain further structural units, preferably those without hydrophilic or hydrophobic side chains or with short side chains, such as, for example, C₁₋₄-alkyl.

In certain cases, it may be helpful to employ a further dispersant, preferably a nonionic surfactant, in addition to the random copolymer. Preferred codispersants are optionally ethoxylated or propoxylated, relatively long-chain alkanols or alkylphenols having various degrees of ethoxylation or propoxylation (for example adducts with from 0 to 50 mol of alkylene oxide; commercially available, for example, from BASF under the trade name Lutensol®).

It may also be advantageous to employ dispersion assistants, preferably water-soluble, high-molecular-weight, organic compounds containing polar groups, such as polyvinylpyrrolidone, copolymers of vinyl propionate or acetate and vinylpyrrolidone, partially saponified copolymers of an acrylate and acrylonitrile, polyvinyl alcohols having various residual acetate contents, cellulose ethers, gelatine, block copolymers, modified starch, low-molecular-weight, carboxyl- and/or sulfonyl-containing polymers, or mixtures of these substances.

Particularly preferred protective colloids are polyvinyl alcohols having a residual acetate content of below 40 mol %, in particular from 5 to 39 mol %, and/or vinylpyrrolidone-vinyl propionate copolymers having a vinyl ester content of below 35% by weight, in particular from 5 to 30% by weight.

The oily phase may advantageously be selected from the following group of substances:
mineral oils, mineral waxes;
oils, such as triglycerides of capric or caprylic acid, furthermore natural oils, such as, for example, castor oil;
organic solvents, such as saturated and unsaturated, cyclic and/or acyclic hydrocarbon compounds, which may optionally contain heteroatoms, such as O, N, S and P;
fats, waxes and other natural and synthetic fatty substances, preferably esters of fatty acids with alcohols having a low carbon number, for example with isopropanol, propylene glycol or glycerol, or esters of fatty alcohols with alkanoic acids having a low carbon number or with fatty acids;
silicone oils, such as dimethylpolysiloxanes, diethylpolysiloxanes, diphenylpolysiloxanes and mixed forms thereof.

For the purposes of the present invention, the oil phase of the emulsions, oleogels or hydrodispersions or lipodispersions is advantageously selected from the group consisting of esters of saturated and/or unsaturated, branched and/or unbranched alkanecarboxylic acids having a chain length of from 3 to 30 carbon atoms and saturated and/or unsaturated, branched and/or unbranched alcohols having a chain length of from 3 to 30 carbon atoms, or from the group consisting of esters of aromatic carboxylic acids and saturated and/or unsaturated, branched and/or unbranched alcohols having a chain length of from 3 to 30 carbon atoms. Ester oils of this type can then advantageously be selected from the group consisting of isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyidodecyl palmitate, oleyl oleate, oleyl erucate, erucyl oleate, erucyl erucate and synthetic, semi-synthetic and natural mixtures of esters of this type, for example jojoba oil.

The oil phase may furthermore advantageously be selected from the group consisting of branched and unbranched hydrocarbons and hydrocarbon waxes, silicone oils, dialkyl ethers, or the group consisting of saturated and unsaturated, branched and unbranched alcohols, and fatty acid triglycerides, specifically the triglycerol esters of saturated and/or unsaturated, branched and/or unbranched alkanecarboxylic acids having a chain length of from 8 to 24, in particular 12-18, carbon atoms. The fatty acid triglycerides may advantageously be selected, for example, from the group consisting of synthetic, semi-synthetic and natural oils, for example olive oil, sunflower oil, soya oil, peanut oil, rapeseed oil, almond oil, palm oil, coconut oil, palm kernel oil and the like.

Any desired mixtures of oil and wax components of this type may also advantageously be employed for the purposes of the present invention. It may also be advantageous to employ waxes, for example cetyl palmitate, as the only lipid component of the oil phase.

The oil phase is advantageously selected from the group consisting of 2-ethylhexyl isostearate, octyidodecanol, isotridecyl isononanoate, iso-eicosane, 2-ethylhexyl cocoate, C₁₂₋₁₅-alkyl benzoate, caprylic/capric acid triglyceride and dicapryl ether.

Particularly advantageous are mixtures of C₁₂₋₁₅-alkyl benzoate and 2-ethylhexyl isostearate, mixtures of C₁₂₋₁₅-alkyl benzoate and isotridecyl isononanoate, as well as mixtures of C₁₂₋₁₅-alkyl benzoate, 2-ethylhexyl isostearate and isotridecyl isononanoate.

Of the hydrocarbons, paraffin oil, squalane and squalene may advantageously be used for the purposes of the present invention.

Furthermore, the oil phase may also advantageously have a content of cyclic or linear silicone oils or consist entirely of oils of this type, although it is preferred to use an additional content of other oil-phase components in addition to the silicone oil or the silicone oils.

The silicone oil to be used in accordance with the invention is advantageously cyclomethicone (octamethylcyclotetrasiloxane). However, it is also advantageous for the purposes of the present invention to use other silicone oils, for example hexamethylcyclotrisiloxane, polydimethylsiloxane or poly(methylphenylsiloxane).

Also particularly advantageous are mixtures of cyclomethicone and isotridecyl isononanoate and of cyclomethicone and 2-ethylhexyl isostearate.

Dispersions which are preferred in accordance with the invention are used as or are paints or coatings, cosmetic or pharmaceutical compositions or cleaning or coating compositions.

Thus, the present invention preferably relates to cosmetic compositions which comprise silica particles and/or silica-coated particulate UV filters and/or silica-encapsulated UV filters. The corresponding materials have already been described above.

Other dispersions which are likewise preferred in accordance with the invention are infrared radiation-curable coatings comprising antimony tin oxide particles. Examples of such particles are the products marketed under the trade name Minatec (Merck).

Suitable dispersion media here are, inter alia, polymers, in particular thermoplastics, such as PE, PP, PVC, PMMA, PS, ABS, polyesters and polyamides. The dispersal can advantageously be carried out by thermal methods (extrusion, compounding) or using solutions of these polymers in suitable solvents.

The following examples are intended to explain the invention in greater detail without limiting it.

### EXAMPLES

### Example 1

### Synthesis of a Random Copolymer of Dodecyl Methacrylate (Lauryl Methacrylate; LMA) and Hydroxyethyl Methacrylate (HEMA)

Control of the molecular weight can be achieved by addition of mercaptoethanol.

LMA and HEMA, in an amount corresponding to Table 1 below, are initially introduced in 12 g of toluene and 300 mg of mercaptoethanol and subjected to free-radical polymerisation for 18 h under argon at 70° C. after initiation of the reaction by addition of 100 mg of AIBN in 1 ml of toluene. Hitherto unreacted residual monomer is likewise polymerised by post-initiation using a further 50 mg of AIBN in 1 ml of toluene and further reaction for 12 h. The solvent is then removed under reduced pressure, and the resultant polymer is dried. The characterisation of the resultant polymers is shown in Table 1.

TABLE 1
Amounts of monomer employed and characterisation
of the resultant polymers

| | | | M_{w} |
|---|---|---|---|
| | LMA [g] | HEMA [g] | [g/mol] |
| E1 | 2.5 | 1.3 | 5800 |
| E2 | 3.8 | 1.3 | 5400 |
| E3 | 2.5 | 0.7 | 5500 |

### Example 2

### Dispersal of SiO₂ Particles

800 mg of the polymer from Example E2 are dissolved in 20 g of paraffin oil. On introduction of 10 g of silica particles (Monospher 1000, Merck; average particle size 1 μm) with stirring (2-blade stirrer; 200/min; no significant increase in viscosity), a stable dispersion is formed.

### Example 3

### Dispersal of SiO₂-Coated TiO₂ Particles

800 mg of the polymer from Example E2 are dissolved in 20 g of cosmetic oil (Miglyol® 8810 N; Condea; INCI: Butylene Glycol Dicaprylate/Dicaprate). On introduction of 10 g of Eusolex® T-AVO (Merck) with stirring (2-blade stirrer; 200/min), a stable dispersion is formed.

### Example 4

### Dispersal of SiO₂-Coated TiO₂ Particles

A dispersion comprising
6% by weight of the polymer from Example E2
57% by weight of Miglyol 8810 N (Condea)
37% by weight of Eusolex® T-AVO (Merck)
   is homogenised using a dispersion disc and subsequently for about 5 minutes using an U-Turrax (at 8000 rpm). After a standing time of one day, the viscosity is about 12,500 mPa s.

### Example 5

### Dispersal of Antimony Tin Oxide Particles

800 mg of the polymer from Example E2 are dissolved in 20 g of terpineol. On introduction of 20 g of antimony tin oxide particles (Minatec®, Merck) with stirring (2-blade stirrer; 200/min; no significant increase in viscosity), a stable dispersion is formed.

### Example 6

### Production of Redispersible, Nanoscale Antimony Tin Oxide Particles

1 g of the polymer from Example E2 is dissolved in 100 g of toluene. 10 g of a stable aqueous dispersion of antimony tin oxide particles (Minatec®, Merck), solids content 2 g, are emulsified therein (U-Turrax, ultrasound). The solvent mixture is removed, giving hydrophobicised particles, which can be redispersed very easily in organic solvents (for example toluene).

### Example 7

### Dispersal of Pyrogenic Silicic Acid

800 mg of the polymer from Example E2 are dissolved in 20 g of toluene. On introduction of 10 g of pyrogenic silicic acid (Aerosilo 50OX; Degussa) with stirring (2-blade stirrer; 200/min; no significant increase in viscosity), a stable dispersion is formed.

1. Use of random copolymers containing at least one structural unit having hydrophobic radicals and at least one structural unit having hydrophilic radicals as dispersants for the preparation of dispersions having incompatible disperse and continuous phases.

2. Use of random copolymers containing at least one structural unit having hydrophobic radicals and at least one structural unit having hydrophilic radicals as dispersants for the dispersal of particles having a hydrophilic surface in oils.

3. Use according to claim 1, characterised in that the hydrophilic particles have a metal (hydr)oxide surface, where the metal (hydr)oxide is preferably selected from oxides and hydroxides of silicon, aluminium, magnesium, antimony, cerium, cobalt, chromium, indium, nickel, zinc, titanium, iron, yttrium, tin, zirconium and mixtures thereof.

4. Use according to claim 1, characterised in that the particles are silica particles or silica-coated particles.

5. Use according to claim 1, characterised in that the dispersant is employed in a concentration of from 0.5 to 80% by weight, preferably in a concentration of from 1 to 50% by weight and particularly preferably in a concentration of from 2 to 8% by weight, based on the dispersion as a whole.

6. Use according to claim 1, characterised in that the particles having a hydrophilic surface are dispersed in a proportion by weight of from 1 to 90% by weight, preferably from 10 to 60% by weight, based on the dispersion.

7. Use according to claim 1, characterised in that at least one further dispersant and/or dispersion assistant is employed.

8. Use according to claim 1, characterised in that the weight ratio of structural units having hydrophobic radicals to structural units having hydrophilic radicals in the random copolymers is in the range from 1:10 to 500:1, preferably in the range from 1:2 to 100:1 and particularly preferably in the range from 1:1 to 10:1.

9. Use according to claim 1, characterised in that the weight average molecular weight of the random copolymers is in the range from M_{w}=1000 to 1,000,000 g/mol, preferably in the range from 2000 to 50,000 g/mol.

10. Use according to at least one of the preceding claims claim 1, characterised in that the copolymers essentially conform to the formula I 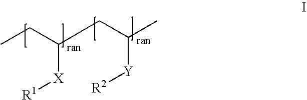
where
X and Y correspond to the radicals of conventional nonionic or ionic monomers, and
R¹ stands for hydrogen or a hydrophobic side group, preferably selected from branched or unbranched alkyl radicals having at least 4 carbon atoms, in which one or more, preferably all, H atoms may have been replaced by fluorine atoms, and
R² stands for a hydrophilic side group, which preferably has one or more phosphonate, phosphate, phosphonium, sulfonate, sulfonium, (quaternary) amine, polyol or polyether radicals, particularly preferably one or more hydroxyl radicals,
ran means that the respective groups in the polymer are arranged in a random distribution,
and where —X—R¹ and —Y—R² may each have a plurality of different meanings within a molecule, and, besides the structural units shown in the formula I, the copolymers may contain further structural units, preferably those with no or with short side chains, such as, for example, C₁₋₄-alkyl.

11. Use according to claim 10, characterised in that X and Y, independently of one another, stand for —O—, —C(═O)—O—, —C(═O)—NH—, —(CH₂)ₙ—, phenylene or pyridyl.

12. Use according to claim 10, characterised in that at least one structural unit of the copolymer contains at least one quaternary nitrogen or phosphorus atom, where R² preferably stands for a —(CH₂)ₘ—(N⁺(CH₃)₂)—(CH₂)ₙ—SO₃⁻ side group or a —(CH₂)ₘ—(N⁺(CH₃)₂)—(CH₂)ₙ—PO₃²⁻, —(CH₂)ₘ—(N⁺(CH₃)₂)—(CH₂)ₙ—O—PO₃²⁻ side group or a —(CH₂)ₘ—(P⁺(CH₃)₂)—(CH₂)ₙ—SO₃⁻ side group, where m stands for an integer from the range from 1 to 30, preferably from the range from 1 to 6, particularly preferably 2, and n stands for an integer from the range from 1 to 30, preferably from the range from 1 to 8, particularly preferably 3.

13. Use according to claim 1, characterised in that the random copolymer employed is a copolymer essentially consisting of lauryl methacrylate (LMA) and hydroxyethyl meth acrylate (HEMA).

14. Use according to claim 1, characterised in that at least one structural unit of the copolymer is an oligomer or polymer, preferably a macromonomer, where polyethers, polyolefins and polyacrylates are particularly preferred as macromonomers.

15. Use according to claim 1, characterised in that at least one structural unit of the copolymer has a phosphonium or sulfonium radical.

16. Use according to claim 1, characterised in that, besides the at least one structural unit having hydrophobic radicals and the at least one structural unit having hydrophilic radicals, the random copolymers contain further structural units, preferably those without hydrophilic or hydrophobic side chains or with short side chains, such as C₁₋₄-alkyl.

17. Oily dispersion comprising hydrophilic particles, characterised in that the dispersant present is at least one random copolymer containing at least one structural unit having hydrophobic radicals and at least one structural unit having hydrophilic radicals.

18. Dispersion according to claim 17, characterised in that the dispersant is present in a concentration of from 0.5 to 80% by weight, preferably in a concentration of from 1 to 50% by weight and particularly preferably in a concentration of from 2 to 8% by weight, based on the dispersion as a whole.

19. Dispersion according to claim 1, characterised in that the hydrophilic particles have a metal (hydr)oxide surface, where the metal (hydr)oxide is preferably selected from oxides and hydroxides of silicon, aluminium, magnesium, antimony, cerium, cobalt, chromium, indium, nickel, zinc, titanium, iron, yttrium, tin, zirconium and mixtures thereof, where the particles are particularly preferably silica particles or silica-coated particles.

20. Dispersion according to claim 1, characterised in that the particles having a hydrophilic surface are present in a proportion by weight of from 1 to 90% by weight, preferably from 10 to 60% by weight, based on the dispersion.

21. Dispersion according to claim 1, characterised in that the dispersion is a cosmetic composition which comprises silica particles and/or silica-coated particulate UV filters.

22. Dispersion according to claim 17, characterised in that the dispersion is an infrared radiation-curable coating comprising antimony tin oxide particles.

23. Process for the preparation of an oily dispersion of hydrophilic particles, characterised in that random copolymers containing at least one structural unit having hydrophobic radicals and at least one structural unit having hydrophilic radicals are mixed with an oil and hydrophilic particles.

24. Process for the preparation of an oily dispersion according to claim 23, characterised in that the random copolymers are initially introduced in an oil, and the hydrophilic particles are subsequently added.

25. Process for the preparation of an oily dispersion according to claim 23, characterised in that an aqueous dispersion of hydrophilic particles is mixed (emulsified) with a solution of a random copolymer in a hydrophobic solvent, and the water or both solvents is/are removed.

26. Process for the preparation of an aqueous dispersion of hydrophobic particles, characterised in that random copolymers containing at least one structural unit having hydrophobic radicals and at least one structural unit having hydrophilic radicals are mixed with water and hydrophobic particles.

27. Process for the preparation of an aqueous dispersion according to claim 26, characterised in that the random copolymers are initially introduced in water, and the hydrophobic particles are subsequently added.

28. Powder composition comprising hydrophilic particles, characterised in that the hydrophilic particles are coated with at least one random copolymer containing at least one structural unit having hydrophobic radicals and at least one structural unit having hydrophilic radicals.

29. Powder composition according to claim 28, characterised in that the hydrophilic particles have a metal (hydr)oxide surface, where the metal (hydr)oxide is preferably selected from oxides and hydroxides of silicon, aluminium, magnesium, antimony, cerium, cobalt, chromium, indium, nickel, zinc, titanium, iron, yttrium, tin, zirconium and mixtures thereof, where the particles are particularly preferably silica particles or silica-coated particles.

30. Powder composition according to claim 1, characterised in that the particles having a hydrophilic surface are present in a proportion by weight of from 20 to 95% by weight, preferably from 30 to 80% by weight, based on the powder composition.

31. Powder composition according to claim 1, characterised in that the hydrophilic particles are essentially silica particles and/or silica-coated particulate UV filters, in particular silica-coated titanium dioxide.

32. Powder composition according to claim 28, characterised in that the hydrophilic particles are essentially antimony tin oxide particles.

33. Process for the preparation of a powder composition, characterised in that a dispersion according to claim 23 is prepared, and the solvent is subsequently removed.

34. Process for the preparation of a dispersion, characterised in that a powder composition according to claim 28 is mixed with at least one oily carrier material.
